(19) United States
(12) Patent Application Publication
(10) Pub. No.: US 2010/0168143 A1
(43) Pub. Date: Jul. 1, 2010

(54) QUINAZOLINE DERIVATIVES

(75) Inventor: Michael Kevin Foote, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (unknown)

(21) Appl. No.: 12/161,121

(22) PCT Filed: Jan. 17, 2007
(86) PCT No.: PCT/GB2007/000119

§ 371 Date: Jul. 16, 2008

(30) Foreign Application Priority Data
Jan. 21, 2006 (GB) 0601215.7

(51) Int. Cl. A61K 31/517; C07D 401/14; A61P 35/00; A61P 35/02

(52) U.S. Cl. 514/266.22; 544/284

(57) ABSTRACT

The invention provided a compound of formula (I) for use in the treatment of disease, in particular proliferative diseases such as cancer and for use in the preparation of medicaments for use in the treatment of proliferative diseases; the invention also processes for the preparation of such compounds, as well as pharmaceutical compositions containing them as active ingredient.

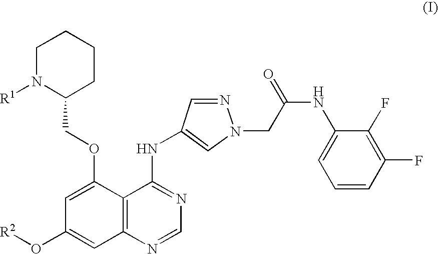

The present invention relates to quinazoline derivatives for use in the treatment of disease, in particular proliferative diseases such as cancer, in the preparation of medicaments for use in the treatment of proliferative diseases, and to processes for their preparation, as well as pharmaceutical compositions containing them as active ingredient.

Cancer (and other hyperproliferative diseases) are characterised by uncontrolled cellular proliferation. This loss of the normal regulation of cell proliferation often appears to occur as the result of genetic damage to cellular pathways that control progress through the to cell cycle.

In eukaryotes, an ordered cascade of protein phosphorylation is thought to control the cell cycle. Several families of protein kinases that play critical roles in this cascade have now been identified. The activity of many of these kinases is increased in human tumours when compared to normal tissue. This can occur by either increased levels of expression of the protein (as a result of gene amplification for example), or by changes in expression of co activators or inhibitory proteins.

The first identified, and most widely studied of these cell cycle regulators have been the cyclin dependent kinases (or CDKs). More recently, protein kinases that are structurally distinct from the CDK family have been identified which play critical roles in regulating the cell cycle and which also appear to be important in oncogenesis. They include the human homologues of the Drosophila aurora and S. cerevisiae Ip11 proteins. The three human homologues of these genes aurora A, aurora B and aurora C (also known as aurora 2, aurora 1 and aurora 3 respectively) encode cell cycle regulated serine-threonine protein kinases (summarised in Adams et al., 2001, Trends in Cell Biology. 11(2): 49-54), which show a peak of expression and kinase activity through G2 and mitosis. Several observations implicate the involvement of human aurora proteins in cancer.

It is known that the aurora A gene maps to chromosome 20q13, a region that is frequently amplified in human tumours including both breast and colon tumours. Aurora A may be the major target gene of this amplicon, since aurora A DNA is amplified and mRNA overexpressed in greater than 50% of primary human colorectal cancers. In these tumours aurora A protein levels appear greatly elevated compared to adjacent normal tissue. In addition, transfection of rodent fibroblasts with human aurora A leads to transformation, conferring the ability to grow in soft agar and form tumours in nude mice (Bischoff et al., 1998, The EMBO Journal. 17(11): 3052-3065). Other work (Zhou et al., 1998, Nature Genetics. 20(2): 189-93) has shown that artificial overexpression of aurora A leads to an increase in centrosome number and an increase in aneuploidy, a known event in the development of cancer.

It has also been shown that there is an increase in expression of aurora B (Adams et al., 2001, Chromsoma. 110(2):65-74) and aurora C (Kimura et al., 1999, Journal of Biological Chemistry, 274(11): 7334-40) in tumour cells when compared to normal cells. Aurora B is also overexpressed in cancer cells and increased levels of aurora B have been shown to correlate with advanced stages of colorectal cancer (Katayama et al (1999) J Natl Cancer Inst. 91:1160). Furthermore, one report suggests that overexpression of aurora B induces aneuploidy through increased phosphorylation of histone H3 at serine 10 and that cells overexpressing aurora B form more aggressive tumours that develop metastases (Ota, T. et al, 2002, Cancer res 62: 5168-5177). Aurora B is a chromosome passenger protein which exists in a stable complex with at least three other passenger proteins, Survivin, INCENP and Borealin (Carmena M et al, 2003, Nat. Rev. Mol. Cell. Biol. 4: 842-854). Survivin is also upregulated in cancer and contains a BIR (Baculovirus Inhibitor of apoptosis protein (IAP) Repeat) domain and may therefore play a role in protecting tumour cells from apoptosis and/or mitotic catastrophe.

With regard to aurora C and its expression is thought to be restricted to the testis although it has been found to be overexpressed in various cancer lines (Katayama H et al, 2003, Cancer and Metastasis Reviews 22: 451-464).

Importantly, it has been demonstrated that abrogation of aurora A expression and function by antisense oligonucleotide treatment of human tumour cell lines (WO 97/22702 and WO 99/37788) leads to cell cycle arrest and exerts an antiproliferative effect in these tumour cell lines. Additionally, small molecule inhibitors of aurora A and aurora B have been demonstrated to have an antiproliferative effect in human tumour cells (Keen et al. 2001, Poster #2455, American Association of Cancer research annual meeting), as has selective abrogation of aurora B expression alone by siRNA treatment (Ditchfield et al., 2003, Journal of Cell Biology, 161(2):267-280). This indicates that inhibition of the function of aurora A and/or aurora B will have an antiproliferative effect that may be useful in the treatment of human tumours and other hyperproliferative diseases. It is believed that inhibition of one or more aurora kinase as a therapeutic approach to such diseases may have significant advantages over targeting signalling pathways upstream of the cell cycle (e.g. those activated by growth factor receptor tyrosine kinases such as epidermal growth factor receptor (EGFR) or other receptors). As the cell cycle is ultimately downstream of all of these diverse signalling events, cell cycle directed therapies such as inhibition of one or more aurora kinase is predicted to be active across all proliferating tumour cells, whilst approaches directed at specific signalling molecules (e.g. EGFR) are believed to be active only in the subset of tumour cells which express those receptors. It is also believed that significant “cross talk” exists between these signalling pathways meaning that inhibition of one component may be compensated for by another.

A number of quinazoline derivatives have already been proposed for use in the inhibition of one or more aurora kinase. WO 04/94410 discloses quinazoline derivatives substituted by a pyrazole ring. These compounds inhibit one or more aurora kinase and are able to inhibit the growth of cells from the human tumour cell line SW620. An example of such a compound is:

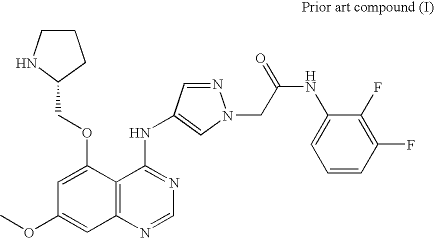

Further more potent compounds having this activity are required and it would be advantageous if such compounds were additionally active in cells known to be resistant to chemotherapeutic agents and in particular in cells that over-express efflux transporters. Examples of efflux transporters include p-glycoprotein, multidrug resistance associated proteins 1, 2, 3, 4 and 5, BCRP, BSEP and sPgp.

It would also be useful if such compounds had more advantageous physical properties that allowed them to be more effectively used in the treatment of hyperproliferative diseases such as cancer.

We have surprisingly found a small selection of compounds, generally a selected subgroup of those described in WO 04/94410, which have superior activity against aurora kinase enzymes, are more potent in cells and in particular in resistant cells. The compounds may also have more advantageous physical properties.

The compounds particularly inhibit the effects of aurora A kinase and/or aurora B kinase and are therefore useful in the treatment of hyperproliferative diseases such as cancer. In particular, the compounds may be used to treat solid or haematological tumours and more particularly any one of, or any combination of, colorectal, breast, lung, prostate, bladder, renal or pancreatic cancer or leukaemia or lymphoma.

Accordingly, one aspect of the invention provides a compound of formula (I)

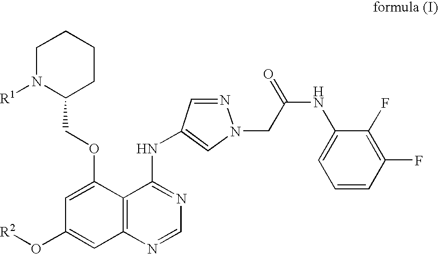

or a salt or prodrug thereof;
wherein R¹ is hydrogen or methyl; and
R² is methyl or ethyl.

As a further aspect a compound of formula (I) or a pharmaceutically acceptable salt thereof is provided.

Compounds of the present invention have been named with the aid of computer software (ACD/Name version 8.0).

Within the present invention it is to be understood that a compound of the invention may exhibit the phenomenon of tautomerism and that the formulae drawings within this specification can represent only one of the possible tautomeric forms. It is to be understood that the invention encompasses any tautomeric form which has aurora kinase inhibitory activity and in particular aurora A and/or aurora B kinase inhibitory activity and is not to be limited merely to any one tautomeric form utilized within the formulae drawings.

It is also to be understood that certain compounds of the invention and salts thereof can exist in solvated as well as unsolvated forms such as, for example, hydrated forms. It is to be understood that the invention encompasses all such solvated forms which have aurora kinase inhibitory activity and in particular aurora A and/or aurora B kinase inhibitory activity.

The present invention relates to the compounds of formula (I) as herein defined as well as to the salts thereof. Salts for use in pharmaceutical compositions will be pharmaceutically acceptable salts, but other salts may be useful in the production of the compounds of formula (I) and their pharmaceutically acceptable salts. Pharmaceutically acceptable salts of the invention may, for example, include acid addition salts of compounds of formula (I) as herein defined which are sufficiently basic to form such salts. Such acid addition salts include but are not limited to furmarate, methanesulphonate, hydrochloride, hydrobromide, citrate and maleate salts and salts formed with phosphoric and sulphuric acid. In addition where compounds of formula (I) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of the formula (I) may be also be administered in the form of a prodrug which is broken down in the human or animal body to give a compound of the formula (I). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);

### c) H. Bundgaard, Advanced Drug Delivery Reviews, 8, 1-38 (1992);

### d) H. Bundgaard, et al., Journal of Pharmaceutical Sciences, 77, 285 (1988); and

### e) N. Kakeya, et al., Chem Pharm Bull, 32, 692 (1984).

In one aspect of the invention R² is methyl. When R² is methyl, the invention provides a compound of formula (IA)

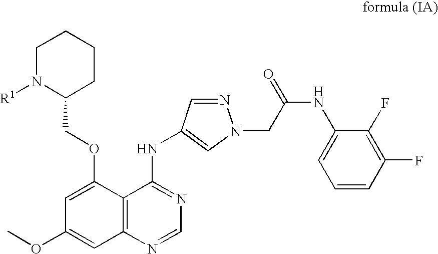

or a salt or prodrug thereof;
wherein R¹ is hydrogen or methyl.

When R² is methyl and R¹ is hydrogen the compound of formula (I) is N-(2,3-difluorophenyl)-2-[4-({7-methoxy-5-[(2R)-piperidin-2-ylmethoxy]quinazolin-4-yl}amino)-1H-pyrazol-1-yl]acetamide. When R² is methyl and R¹ is methyl the compound of formula (I) is N-(2,3-difluorophenyl)-2-[4-({7-methoxy-5-[(2R)-1-methylpiperidin-2-ylmethoxy]quinazolin-4-yl} amino)-1H-pyrazol-1-yl]acetamide.

In one aspect of the invention R² is ethyl. When R² is ethyl, the invention provides a compound of formula (IB)

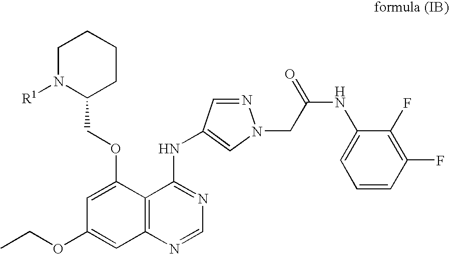

or a salt or prodrug thereof;
wherein R¹ is hydrogen or methyl.

When R² is ethyl and R¹ is hydrogen the compound of formula (I) is N-(2,3-difluorophenyl)-2-[4-({7-ethoxy-5-[(2R)-piperidin-2-ylmethoxy]quinazolin-4-yl}amino)-1H-pyrazol-1-yl]acetamide. When R² is ethyl and R¹ is methyl the compound of formula (I) is N-(2,3-difluorophenyl)-2-[4-({7-ethoxy-5-[(2R)-1-methylpiperidin-2-ylmethoxy]quinazolin-4-yl}amino)-1H-pyrazol-1-yl]acetamide.

The present invention also provides a process for the preparation of a compound of formula (I) where R¹ is methyl, which process comprises reacting a compound of formula (I) where R¹ is hydrogen with formaldehyde in formic acid at elevated temperatures from 50° C. to 100° C. for 30 minutes to 2 hours, and thereafter if necessary:

i) removing any protecting groups; and/or
ii) forming a salt or prodrug thereof.

A process for the preparation of a compound of formula (I) where R¹ is hydrogen comprises reacting a compound of formula (II):

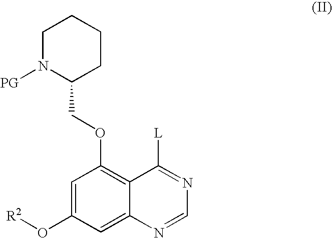

where PG is a suitable protecting group such as tert-butoxycarbonyl (BOC), benzyloxycarbonyl (Z) or 9-fluorenylmethyloxycarbonyl (Fmoc) and L is a suitable leaving group such as chloro, with 2-(4-amino-1H-pyrazol-1-yl)-N-(2,3-difluorophenyl)acetamide and thereafter if necessary:
i) removing any protecting groups; and/or
ii) forming a salt or prodrug thereof.

This reaction may be performed under a range of conditions described in the literature such as reacting a compound of formula (II) with 2-(4-amino-1H-pyrazol-1-yl)-N-(2,3-difluorophenyl)acetamide in a solvent such as isopropanol or dimethylacetamide, with or without an acid catalyst such as hydrochloric acid, at a temperature of 20 to 100° C. for 30 minutes to 24 hours.

The invention further provides a process for the preparation of a compound of formula (II) which process comprises the reaction of a compound of formula (III):

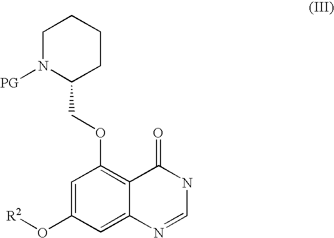

where PG is a suitable protecting group such as tert-butoxycarbonyl (BOC), benzyloxycarbonyl (Z) or 9-fluorenylmethyloxycarbonyl (Fmoc) with a suitable chlorinating agent such as phosphorus oxychloride in a suitable solvent such as 1,2-dichloroethane or acetonitrile in the presence of a suitable base such as di-iso-propylethyl amine, at a temperature of 0 to 80° C. for 2 to 24 hours.

This process may further comprise a process for the preparation of a compound of formula (III) where PG is a suitable protecting group such as tert-butoxycarbonyl (BOC), benzyloxycarbonyl (Z) or 9-fluorenylmethyloxycarbonyl (Fmoc) which process comprises the reaction of a compound of formula (IV)

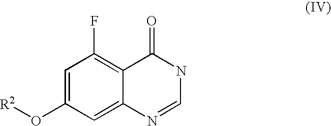

with a compound of formula (V) wherein PG¹ is hydrogen or a suitable protecting group such as benzyl:

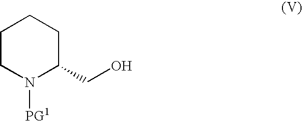

with subsequent protecting group transformation.

This reaction may be performed under a range of conditions described in the literature, for example in a solvent such as tetrahydrofuran, dimethylformamide, dimethylacetamide or 1-methyl-2-pyrrolidinone with a base such as sodium hydride or potassium tert-butoxide at a temperature of 20 to 100° C. for 2 to 24 hours.

Compounds of formula (V) where PG¹ is either hydrogen or a suitable protecting group such as benzyl are either known in the art or can be derived from other compounds known in the art by conventional methods which would be apparent from the literature.

A compound of formula (IV) may be prepared by the reaction of a compound of formula (VI)

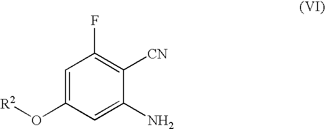

with formic acid with a catalytic amount of a mineral acid such as sulphuric acid at a temperature such as 100° C. for 2 to 24 hours.

Compounds of formula (VI) are either known in the art, can be derived from other compounds known in the art by conventional methods that would be apparent from the literature, or can be prepared as described in the Examples.

The invention also provides a process for the preparation of 2-(4-amino-1H-pyrazol-1-yl)-N-(2,3-difluorophenyl)acetamide which process comprises the hydrolysis of N-(2,3-difluorophenyl)-2-{4-[(diphenylmethylene)amino]-1H-pyrazol-1-yl}acetamide in a suitable solvent such as ethyl acetate in the presence of a concentrated aqueous acid such as hydrochloric acid.

N-(2,3-difluorophenyl)-2-{4-[(diphenylmethylene)amino]-1H-pyrazol-1-yl}acetamide may be prepared by a process which comprises the reaction of a compound of formula (VII):

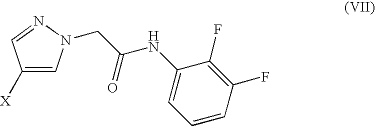

where X is a halogen such as bromo or iodo with benzophenone imine. This reaction may be performed under a range of conditions described in the literature such as heating a compound of formula (VII) with benzophenone imine in a solvent such as 1,4-dioxane, in the presence of a suitable base catalyst such as sodium tert-butoxide, cesium carbonate, potassium carbonate or potassium phosphate, in the presence of a suitable ligand such as (9,9-dimethyl-9H-xanthene-4,5-diyl)bis(diphenylphosphine), 1,1′-binaphthalene-2,2′-diylbis(diphenylphosphine) or 1,1′-bis(diphenylphosphino)ferrocene, and in the presence of a suitable catalyst such as tris(dibenzylideneacetone)dipalladium(0) or palladium acetate, at a temperature such as 90° C. for 30 minutes to 6 hours.

This process may further comprise a process for the preparation of a compound of formula (VII) which process comprises the reaction of a compound of formula (VIII):

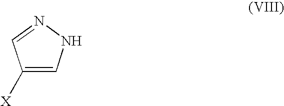

where X is a halogen such as bromo or iodo with a compound of formula (IX):

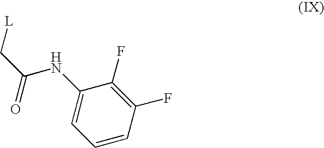

where L is a leaving group such as chloro or bromo. This reaction may be performed under a range of conditions described in the literature such as reacting a compound of formula (VIII) with a compound of formula (IX), in the presence of a base such as potassium carbonate, in a solvent such as dimethylacetamide at a temperature such as 20° C. for 14 to 48 hours.

Compounds of formula (VIII) and (IX) are either known in the art or can be derived from other compounds known in the art by conventional methods which would be apparent from the literature.

Alternatively, 2-(4-amino-1H-pyrazol-1-yl)-N-(2,3-difluorophenyl)acetamide may be prepared by the reduction of N-(2,3-difluorophenyl)-2-(4-nitro-1H-pyrazol-1-yl)acetamide. This reaction may be performed under a range of conditions described in the literature such as reducing N-(2,3-difluorophenyl)-2-(4-nitro-1H-pyrazol-1-yl)acetamide under an atmosphere of hydrogen at a pressure of 1 to 5 bar, in the presence of a suitable catalyst such as platinum oxide or palladium on carbon in a suitable solvent such as ethanol and/or ethylacetate at a suitable temperature such as 20° C. for 0.5 to 5 hours.

N-(2,3-difluorophenyl)-2-(4-nitro-1H-pyrazol-1-yl)acetamide may be prepared by the reaction of (4-nitro-1H-pyrazol-1-yl)acetic acid with 2,3-difluoroaniline. This reaction may be performed under a range of conditions described in, the literature such as coupling (4-nitro-1H-pyrazol-1-yl)acetic acid with 2,3-difluoroaniline with phosphorus oxychloride and pyridine in a solvent such as dichloromethane at a temperature of 0 to 20° C. for 2-3 hours.

(4-nitro-1H-pyrazol-1-yl)acetic acid and 2,3-difluoroaniline are known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of allylthio to alkylsulphinyl or alkylsulphonyl.

It will also be appreciated that in some of the reactions mentioned herein it may be to necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tent-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

According to a further aspect of the invention there is provided a pharmaceutical composition which comprises a compound formula (I), or a pharmaceutically acceptable salt or prodrug thereof, as defined herein in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algesic acid; binding agents such as starch; lubricating agents such as magnesium stearate, stearic acid or talc; preservative agents such as ethyl or propyl g-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, soya bean oil, coconut oil, or preferably to olive oil, or any other acceptable vehicle.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl g-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible or lyophilised powders and granules suitable for preparation of an aqueous suspension or solution by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, solutions, emulsions or particular systems, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in polyethylene glycol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30 μm or much less preferably 5 μm or less and more preferably between 5 μm and 1 μm, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

Therefore in a further aspect of the invention there is provided a compound of formula (I), or a pharmaceutically acceptable salt or prodrug thereof, for use in therapy. Further provided is a compound of formula (I), or a pharmaceutically acceptable salt or prodrug thereof, for use as a medicament. Another aspect of the invention provides a compound of formula (I), or a pharmaceutically acceptable salt or prodrug thereof, for use as a medicament for the treatment of hyperproliferative diseases such as cancer and in particular, for the treatment of any one of or any combination of, colorectal, breast, lung, prostate, bladder, renal or pancreatic cancer or leukaemia or lymphoma.

Additionally a compound of formula (I), or a pharmaceutically acceptable salt or prodrug thereof is provided for use in a method of treatment of a warm-blooded animal such as man by therapy. Another aspect of the invention provides a compound of formula (I), or a pharmaceutically acceptable salt or prodrug thereof, for use in a method of treatment of hyperproliferative diseases such as cancer and in particular, for the treatment of any one of or any combination of, colorectal, breast, lung, prostate, bladder, renal or pancreatic cancer or leukaemia or lymphoma.

In another aspect of the invention, there is provided the use of a compound of formula (I) or a pharmaceutically acceptable salt or prodrug thereof, in the preparation of a medicament for the treatment of a disease where the inhibition of one or more aurora kinase(s) is beneficial. In particular it is envisaged that inhibition of aurora A kinase and/or aurora B kinase may be beneficial. Preferably inhibition of aurora B kinase is beneficial. In another aspect of the invention, there is provided the use of a compound of formula (I) or a pharmaceutically acceptable salt or prodrug thereof, in the preparation of a medicament for the treatment of hyperproliferative diseases such as cancer and in particular, for the treatment of any one of or any combination of, colorectal, breast, lung, prostate, bladder, renal or pancreatic cancer or leukaemia or lymphoma.

According to yet another aspect, there is provided a compound of formula (I) or a pharmaceutically acceptable salt, ester or prodrug thereof for use in the method of treating a human suffering from a disease in which the inhibition of one or more aurora kinase is beneficial, comprising the steps of administering to a person in need thereof a therapeutically effective amount of a compound of formula (I) or a pharmaceutically acceptable salt or prodrug thereof. In particular it is envisaged that inhibition of aurora A kinase and/or aurora B kinase may be beneficial. Preferably inhibition of aurora B kinase is beneficial. Further provided is a compound of formula (I) or a pharmaceutically acceptable salt or prodrug thereof for use in the method of treating a human suffering from a hyperproliferative disease such as cancer and in particular, any one of or any combination of, colorectal, breast, lung, prostate, bladder, renal or pancreatic cancer or leukaemia or lymphoma, comprising the steps of administering to a person in need thereof a therapeutically effective amount of a compound of formula (I) or a pharmaceutically acceptable salt or prodrug thereof. The use of a compound of formula (I) or a pharmaceutically acceptable salt or prodrug thereof in any of the methods of treating a human described above also form aspects of this invention.

For the above mentioned therapeutic uses the dose administered will vary with the compound employed, the mode of administration, the treatment desired, the disorder indicated and the age and sex of the animal or patient. The size of the dose would thus be calculated according to well known principles of medicine.

In using a compound of formula (I) for therapeutic or prophylactic purposes it will generally be administered so that a daily dose in the range, for example, 0.05 mg/kg to 50 mg/kg body weight (and in particular 0.05 mg/kg to 15 mg/kg body weight) is received, given if required in divided doses. In general lower doses will be administered when a parenteral route is employed. Thus, for example, for intravenous administration, a dose in the range, for example, 0.05 mg/kg to 25 mg/kg body weight (and in particular 0.05 mg/kg to 15 mg/kg body weight) will generally be used. Similarly, for administration by inhalation, a dose in the range, for example, 0.05 mg/kg to 25 mg/kg body weight (and in particular 0.05 mg/kg to 15 mg/kg body weight) will be used.

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of
5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino} thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin ανβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

In addition a compound of the invention or a pharmaceutically acceptable salt or prodrug thereof, may be used in combination with one or more cell cycle inhibitors. In particular with cell cycle inhibitors which inhibit bub1, bubR1 or CDK.

Such conjoint treatment may be achieved by way of the simultaneous, sequential or separate dosing of the individual components of the treatment. Such combination products employ the compounds of this invention within the dosage range described herein and the other pharmaceutically-active agent within its approved dosage range.

In addition to their use in therapeutic medicine, a compound of formula (I) and a pharmaceutically acceptable salt or prodrug thereof are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

The compounds of the invention inhibit the serine-threonine kinase activity of the aurora kinases, in particular aurora A kinase and/or aurora B kinase and thus inhibit the cell cycle and cell proliferation. Compounds which inhibit aurora B kinase are of particular interest. The compounds are also active in resistant cells and have advantageous physical properties. These properties may be assessed for example, using one or more of the procedures set out below.

### (a) In Vitro Aurora A Kinase Inhibition Test

This assay determines the ability of a test compound to inhibit serine-threonine kinase activity. DNA encoding aurora A may be obtained by total gene synthesis or by cloning. This DNA may then be expressed in a suitable expression system to obtain polypeptide with serine-threonine kinase activity. In the case of aurora A, the coding sequence was isolated from cDNA by polymerase chain reaction (PCR) and cloned into the BamH1 and Not1 restriction endonuclease sites of the baculovirus expression vector pFastBac HTc (GibcoBRL/Life technologies). The 5′ PCR primer contained a recognition sequence for the restriction endonuclease BamH1 5′ to the aurora A coding sequence. This allowed the insertion of the aurora A gene in frame with the 6 histidine residues, spacer region and rTEV protease cleavage site encoded by the pFastBac HTc vector. The 3′ PCR primer replaced the aurora A stop codon with additional coding sequence followed by a stop codon and a recognition sequence for the restriction endonuclease Not1. This additional coding sequence (5′ TAC CCA TAC GAT GTT CCA GAT TAC GCT TCT TAA 3′) encoded for the polypeptide sequence YPYDVPDYAS. This sequence, derived from the influenza hemagglutin protein, is frequently used as a tag epitope sequence that can be identified using specific monoclonal antibodies. The recombinant pFastBac vector therefore encoded for an N-terminally 6 his tagged, C terminally influenza hemagglutin epitope tagged Aurora-A protein. Details of the methods for the assembly of recombinant DNA molecules can be found in standard texts, for example Sambrook et al. 1989, Molecular Cloning—A Laboratory Manual, 2^{nd} Edition, Cold Spring Harbor Laboratory press and Ausubel et al. 1999, Current Protocols in Molecular Biology, John Wiley and Sons Inc.

Production of recombinant virus can be performed following manufacturer's protocol from GibcoBRL. Briefly, the pFastBac-1 vector carrying the aurora A gene was transformed into E. coli DH10Bac cells containing the baculovirus genome (bacmid DNA) and via a transposition event in the cells, a region of the pFastBac vector containing gentamycin resistance gene and the aurora A gene including the baculovirus polyhedrin promoter was transposed directly into the bacmid DNA. By selection on gentamycin, kanamycin, tetracycline and X-gal, resultant white colonies should contain recombinant bacmid DNA encoding aurora A. Bacmid DNA was extracted from a small scale culture of several BH10Bac white colonies and transfected into Spodoptera frugiperda Sf21 cells grown in TC100 medium (GibcoBRL) containing 10% serum using CellFECTIN reagent (GibcoBRL) following manufacturer's instructions. Virus particles were harvested by collecting cell culture medium 72 hours post transfection. 0.5 ml of medium was used to infect 100 ml suspension culture of Sf21s containing 1×10⁷ cells/ml. Cell culture medium was harvested 48 hours post infection and virus titre determined using a standard plaque assay procedure. Virus stocks were used to infect SD and “High 5” cells at a multiplicity of infection (MOI) of 3 to ascertain expression of recombinant aurora A protein.

For the large scale expression of aurora A kinase activity, Sf21 insect cells were grown at 28° C. in TC100 medium supplemented with 10% foetal calf serum (Viralex) and 0.2% F68 Pluronic (Sigma) on a Wheaton roller rig at 3 r.p.m. When the cell density reached 1.2×10⁶ cells ml⁻¹ they were infected with plaque-pure aurora A recombinant virus at a multiplicity of infection of 1 and harvested 48 hours later. All subsequent purification steps were performed at 4° C. Frozen insect cell pellets containing a total of 2.0×10⁸ cells were thawed and diluted with lysis buffer (25 mM HEPES (N-[2-hydroxyethyl]piperazine-N′-[2-ethanesulphonic acid]) ph7.4 at 4° C., 100 mM KCl, 25 mM NaF, 1 mM Na₃VO₄, 1 mM PMSF (phenylmethylsulphonyl fluoride), 2 mM 2-mercaptoethanol, 2 mM imidazole, 1 μg/ml aprotinin, 1 μg/ml pepstatin, 1 μg/ml leupeptin), using 1.0 ml per 3×10⁷ cells. Lysis was achieved using a dounce homogeniser, following which the lysate was centrifuged at 41,000 g for 35 minutes. Aspirated supernatant was pumped onto a 5 mm diameter chromatography column containing 500 μl Ni NTA (nitrilo-tri-acetic acid) agarose (Qiagen, product no. 30250) which had been equilibrated in lysis buffer. A baseline level of UV absorbance for the eluent was reached after washing the column with 12 ml of lysis buffer followed by 7 ml of wash buffer (25 mM HEPES ph7.4 at 4° C., 100 mM KCl, 20 mM imidazole, 2 mM 2-mercaptoethanol). Bound aurora A protein was eluted from the column using elution buffer (25 mM HEPES pH7.4 at 4° C., 100 mM KCl, 400 mM imidazole, 2 mM 2-mercaptoethanol). An elution fraction (2.5 ml) corresponding to the peak in UV absorbance was collected. The elution fraction, containing active aurora A kinase, was dialysed exhaustively against dialysis buffer (25 mM HEPES pH7.4 at 4° C., 45% glycerol (v/v), 100 mM KCl, 0.25% Nonidet P40 (v/v), 1 mM dithiothreitol).

Each new batch of aurora A enzyme was titrated in the assay by dilution with enzyme diluent (25 mM Tris-HCl pH7.5, 12.5 mM KCl, 0.6 mM DTT). For a typical batch (supplied by Upstate), stock enzyme is diluted 1 μm per ml with enzyme diluent and 20 μl of dilute enzyme is used for each assay well. Test compounds (at 10 mM in dimethylsulphoxide (DMSO) were diluted with water and 10 μl of diluted compound was transferred to wells in the assay plates. “Total” and “blank” control wells contained 2.5% DMSO instead of compound. 20 μl of freshly diluted enzyme was added to all wells, apart from “blank” wells. 20 μl of enzyme diluent was added to “blank” wells. 20 μl of reaction mix (25 mM Tris-HCl, 78.4 mM KCl, 2.5 mM NaF, 0.6 mM dithiothreitol, 6.25 mM MnCl₂, 25 mM ATP, 7.5 μM peptide substrate [biotin-LRRWSLGLRRWSLGLRRWSLGLRRWSLG]) containing 0.2 μCi [γ³³P]ATP (Amersham Pharmacia, specific activity≧2500Ci/mmol) was then added to all test wells to start the reaction. The plates were incubated at room temperature for 60 minutes. To stop the reaction 100 μl 20% v/v orthophosphoric acid was added to all wells. The peptide substrate was captured on positively-charged nitrocellulose P30 filtermat (Whatman) using a 96-well plate harvester (TomTek) and then assayed for incorporation of ³³P with a Beta plate counter. “Blank” (no enzyme) and “total” (no compound) control values were used to determine the dilution range of test compound which gave 50% inhibition of enzyme activity (IC50 values). The compounds of the invention generally give IC50 values of 0.1 nM to 5 μM.

### (b) In Vitro Aurora B Kinase Inhibition Test

This assay determines the ability of a test compound to inhibit serine-threonine kinase activity. DNA encoding aurora B may be obtained by total gene synthesis or by cloning. This DNA may then be expressed in a suitable expression system to obtain polypeptide with serine-threonine kinase activity. In the case of aurora B, the coding sequence was isolated from cDNA by polymerase chain reaction (PCR) and cloned into the pFastBac system in a manner similar to that described above for aurora A (i.e. to direct expression of a 6-histidine tagged aurora B protein).

For the large scale expression of aurora B kinase activity, Sf21 insect cells were grown at 28° C. in TC100 medium supplemented with 10% foetal calf serum (Viralex) and 0.2% F68 Pluronic (Sigma) on a Wheaton roller rig at 3 r.p.m. When the cell density reached 1.2×10⁶ cells ml⁻¹ they were infected with plaque-pure aurora B recombinant virus at a multiplicity of infection of 1 and harvested 48 hours later. All subsequent purification steps were performed at 4° C. Frozen insect cell pellets containing a total of 2.0×10⁸ cells were thawed and diluted with lysis buffer (50 mM HEPES (N-[2-hydroxyethyl]piperazine-N′-[2-ethanesulphonic acid]) pH7.5 at 4° C., 1 mM Na₃VO₄, 1 mM PMSF (phenylmethylsulphonyl fluoride), 1 mM dithiothreitol, 1 μg/ml aprotinin, 1 μg/ml pepstatin, 1 μg/ml leupeptin), using 1.0 ml per 2×10⁷ cells. Lysis was achieved using a sonication homogeniser, following which the lysate was centrifuged at 41,000 g for 35 minutes. Aspirated supernatant was pumped onto a 5 mm diameter chromatography column containing 1.0 ml CM sepharose Fast Flow (Amersham Pharmacia Biotech) which had been equilibrated in lysis buffer. A baseline level of UV absorbance for the eluent was reached after washing the column with 12 ml of lysis buffer followed by 7 ml of wash buffer (50 in M HEPES 017.4 at 4° C., 1 mM dithiothreitol). Bound aurora B protein was eluted from the column using a gradient of elution buffer (50 mM HEPES pH7.4 at 4° C., 0.6 M NaCl, 1 mM dithiothreitol, running from 0% elution buffer to 100% elution buffer over 15 minutes at a flowrate of 0.5 ml/min). Elution fractions (1.0 ml) corresponding to the peak in UV absorbance was collected. Elution fractions were dialysed exhaustively against dialysis buffer (25 mM HEPES pH7.4 at 4° C., 45% glycerol (v/v), 100 mM KCl, 0.05% (v/v) IGEPAL CA630 (Sigma Aldrich), 1 mM dithiothreitol). Dialysed fractions were assayed for aurora B kinase activity.

Aurora B-INCENP enzyme (supplied by Upstate) was prepared by activating aurora B (5 μM)_ in 50 mM Tris-HCl pH 7.5, 0.1 mM EGTA, 0.1% 2-mercaptoethanol, 0.1 mM sodium vandate, 10 mM magnesium acetate, 0.1 mM ATP with 0.1 mg/ml GST-INCEPT [826-919] at 30° C. for 30 minutes.

Each new batch of aurora B-INCENP enzyme was titrated in the assay by dilution with enzyme diluent (25 mM Tris-HCl pH7.5, 12.5 mM KCl, 0.6 mM DTT). For a typical batch, stock enzyme is diluted 1 in 40 with enzyme diluent and 20 μl of dilute enzyme is used for each assay well. Test compounds (at 10 mM in dimethylsulphoxide (DMSO) were diluted with water and 10 μl of diluted compound was transferred to wells in the assay plates. “Total” and “blank” control wells contained 2.5% DMSO instead of compound. 20 μl of freshly diluted enzyme was added to all wells, apart from “blank” wells. 20 μl of enzyme diluent was added to “blank” wells. 20 μl of reaction mix (25 mM Tris-HCl, 12.7 mM KCl, 2.5 mM NaF, 0.6 mM dithiothreitol, 6.25 mM MnCl₂, 15 mM ATP, 6.25 μM peptide substrate [biotin-LRRWSLGLRRWSLGLRRWSLGLRRWSLG]) containing 0.2 μCi [γ³³P]ATP (Amersham Pharmacia, specific activity≧2500Ci/mmol) was then added to all test wells to start the reaction. The plates were incubated at room temperature for 60 minutes. To stop the reaction 100 μl 20% v/v orthophosphoric acid was added to all wells. The peptide substrate was captured on positively-charged nitrocellulose P30 filtermat (Whatman) using a 96-well plate harvester (TomTek) & then assayed for incorporation of ³³P with a Beta plate counter. “Blank” (no enzyme) and “total” (no compound) control values were used to determine the dilution range of test compound which gave 50% inhibition of enzyme activity (1050 values). The compounds of the invention generally give IC50 values of 0.05 nM to 0.5 μM, particularly 0.05 nM to 2 nM. In particular compound 1 has an IC50 value of 1.4 nM and compound 2 has an IC50 value of 1.4 nM.

### (c) In Vitro Cell Phenotype and Substrate Phosphorylation Assay

This assay is used to determine the cellular effects of compounds on SW620 human colon tumour cells in vitro. Compounds typically cause inhibition of levels of phosphohistone H3 and an increase in the nuclear area of the cells.

10⁴ SW620 cells per well were plated in 100 μl DMEM media (containing 10% FCS and 1% glutamine) (DMEM is Dulbecco's Modified Eagle's Medium (Sigma D6546)) in costar 96 well plates and left overnight at 37° C. and 5% CO₂ to adhere. The cells were then dosed with compound diluted in media (50 μl is added to each well to give 0.00015μ-1 μM concentrations of compound) and after 24 hours of treatment with compound, the cells were fixed.

The cells were first examined using a light microscope and any cellular changes in morphology were noted. 100 μl of 3.7% formaldehyde was then added to each well, and the plate was left for at least 30 minutes at room temperature. Decanting and tapping the plate on a paper towel removed the fixative and plates were then washed once in PBS (Dulbecco's Phosphate Buffered Saline (Sigma D8537)) using an automated plate washer. 100 μl PBS and 0.5% triton X-100 was added and the plates were put on a shaker for 5 minutes. The plates were washed in 100 μl PBS and solution tipped off. 50 μl of primary antibody, 1:500 rabbit anti-phosphohistone H3 in PBS 1% BSA (bovine serum albumin) and 0.5% tween, was added. Anti-phosphohistone H3 rabbit polyclonal 06-750 was purchased from Upstate Biotechnology. The plates were left 1 hour at room temperature on a shaker.

The next day, the antibody was tipped off and the plates were washed twice with PBS. In an unlit area, 50 μl of secondary antibody, 1:10,000 Hoechst and 1:200 Alexa Fluor 488 goat anti rabbit IgGA (cat no. 11008 molecular probes) in PBS 1% BSA, 0.5% tween was added. The plates were wrapped in tin foil and shaken for 1 hour at room temperature. The antibody was tipped off and plates were washed twice with PBS. 200 μl PBS was added to each well, and the plates were shaken for 10 minutes, PBS was removed. 100 μPBS was added to each well and the plates were sealed ready to analyse. Analysis was carried out using an Arrayscan Target Activation algorithm to measure cellular levels of phosphohistone H3 and changes in nuclear area. Results were reported as the effective concentration required to give 50% inhibition of phosphohistone 1-13 levels and similarly for a 50% increase in nuclear area of cells (EC50 values). The compounds of the invention generally give EC50 values for inhibition of phosphohistone H3 levels of 0.5 nM to 0.1 μM.

### (d) In Vitro Drug-Resistant Cell Phenotype and Substrate Phosphorylation Assay.

This assay is used to determine the cellular effects of compounds on drug-resistant MCF7-ADR human breast tumour cells in vitro.

MCF7 cells were pretreated with multiple doses of adriomycin (Dr. Hickinson, Molecular Oncology lab, ICRF, University of Oxford Institute of Molecular Medicine, Headington, Oxford), a procedure that resulted in overexpression of drug-resistant proteins by the cells. Compounds typically cause inhibition of levels of phosphohistone H3 and an increase in the nuclear area of treated cells. However, if the compounds are substrates of the overexpressed efflux proteins, they will appear less active in this assay than in the previous SW620 assay.

0.8×10⁴ MCF7-ADR cells per well were plated in 100 μl DMEM media (containing 10% FCS (foetal calf serum) and 1% glutamine) in costar 96 well plates and left overnight at 37° C. and 5% CO₂ to adhere.

All other procedures are identical to those for the above assay using SW620 cells.

The compounds of the invention generally have EC50 values for inhibition of phosphohistone H3 levels of 0.05 nM to 0.1 μM, particularly 0.5 nM to 1 μM and more particularly 0.5 nM to 0.5 μM. In particular compound 1 has an EC50 value of less than 1 nM and compound 2 has an EC50 value of 20 nM. Prior art compound (I) has been found to have an EC50 value of 120 nM.

The invention will now be illustrated in the following examples, in which standard techniques known to the skilled chemist and techniques analogous to those described in these examples may be used where appropriate, and in which, unless otherwise stated:

(i) evaporations were carried out by rotary evaporation in vacuo and work up procedures were carried out after removal of residual solids such as drying agents by filtration;
(ii) operations were carried out at ambient temperature, typically in the range 18-25° C. and in air unless stated, or unless the skilled person would otherwise operate under an atmosphere of an inert gas such as argon;
(iii) column chromatography (by the flash procedure) and medium pressure liquid chromatography (MPLC) were performed on Merck Kieselgel silica (Art. 9385);
(iv) yields are given for illustration only and are not necessarily the maximum attainable;
(v) the structures of the end products of the formula (I) were generally confirmed by nuclear (generally proton) magnetic resonance (NMR) and mass spectral techniques; proton magnetic resonance chemical shift values were measured in deuterated dimethyl sulphoxide (DMSO d₆) (unless otherwise stated) on the delta scale (ppm downfield from tetramethylsilane) using one of the following four instruments

Varian Gemini 2000 spectrometer operating at a field strength of 300 MHz

Bruker DPX300 spectrometer operating at a field strength of 300 MHz

JEOL EX 400 spectrometer operating at a field strength of 400 MHz

Bruker Avance 500 spectrometer operating at a field strength of 500 MHz

Peak multiplicities are shown as follows: s, singlet; d, doublet; dd, double doublet; t, triplet; q, quartet; qu, quintet; m, multiplet; br s, broad singlet;
(vi) robotic synthesis was carried out using a Zymate XP robot, with solution additions via a Zymate Master Laboratory Station and stirred via a Stem RS5000 Reacto-Station at 25° C.;
(vii) work up and purification of reaction mixtures from robotic synthesis was carried out as follows: evaporations were carried out in vacuo using a Genevac HT 4; column chromatography was performed using either an Anachem Sympur MPLC system on silica using 27 mm diameter columns filled with Merck silica (60 μm, 25 g); the structures of the final products were confirmed by LCMS (liquid chromatography mass spectrometry) on a Waters 2890/ZMD micromass system using the following and are quoted as retention time (RT) in minutes:
Column: waters symmetry C18 3.5 μm 4.6×50 mm

### Solvent A: H₂O

### Solvent B: CH₃CN

### Solvent C: MeOH+5% HCOOH

Flow rate: 2.5 ml/min
Run time: 5 minutes with a 4.5 minute gradient from 0-100% C
Wavelength: 254 nm, bandwidth 10 nm
Mass detector: ZMD micromass
Injection volume 0.005 ml
(viii) Analytical LCMS for compounds which had not been prepared by robotic synthesis was performed on a Waters Alliance HT system using the following and are quoted as retention time (RT) in minutes:
Column: 2.0 mm×5 cm Phenomenex Max-RP 80A

### Solvent A: Water

### Solvent B: Acetonitrile

Solvent C: Methanol/1% formic acid or Water/1% formic acid
Flow rate: 1.1 ml/min
Run time: 5 minutes with a 4.5 minute gradient from 0-95% B+constant 5% solvent C
Wavelength: 254 nm, bandwidth 10 nm
Injection volume 0.005 ml
Mass detector: Micromass ZMD
(ix) Preparative high performance liquid chromatography (HPLC) was performed on either

Waters preparative LCMS instrument, with retention time (RT) measured in minutes:

Column: β-basic Hypercil (21×100 mm) 5 μm
Solvent A: Water/0.1% Ammonium carbonate

### Solvent B: Acetonitrile

Flow rate: 25 ml/min
Run time: 10 minutes with a 7.5 minute gradient from 0-100% B
Wavelength: 254 nm, bandwidth 10 nm
Injection volume 1-1.5 ml
Mass detector: Micromass ZMD

Gilson preparative HPLC instrument, with retention time (RT) measured in minutes:

Column: 21 mm×15 cm Phenomenex Luna2 C18
Solvent A: Water+0.1% trifluoracetic acid,
Solvent B: Acetonitrile+0.1% trifluoracetic acid
Flow rate: 21 ml/min
Run time: 20 minutes with various 10 minute gradients from 5-100% B
Wavelength: 254 nm, bandwidth 10 nm
Injection volume 0.1-4.0 ml
(x) intermediates were not generally fully characterised and purity was assessed by thin layer chromatography (TLC), HPLC, infra-red (IR), MS or NMR analysis.

TABLE 1

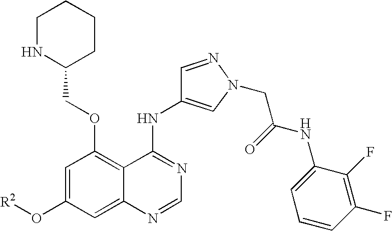

| Compound | R² |
|---|---|
| 1 | Et |
| 2 | Me |

### EXAMPLE 1

### Preparation of Compound 1 in Table 1—N-(2,3-difluorophenyl)-2-[4-{7-ethoxy-5-[(2R)-piperidin-2-ylmethoxy]quinazolin-4-yl}amino)-1H-pyrazol-1-yl]acetamide

To a solution of tert-butyl (2R)-2-[({4-[(1-{2-[(2,3-difluorophenyl)amino]-2-oxoethyl}-1H-pyrazol-4-yl)amino]-7-ethoxyquinazolin-5-yl}oxy)methyl]piperidine-1-carboxylate hydrochloride (400 mg, 0.59 mmol) in dichloromethane (10 ml) was added trifluoroacetic acid (2.5 ml) and the reaction mixture stirred at room temperature for 30 minutes. The resulting solution was evaporated and the residue mixed with 5% aqueous sodium carbonate solution (40 ml) and then extracted with 10% methanol in dichloromethane (2×40 ml). The combined organic extracts were dried over magnesium sulphate and then evaporated. The residue was purified by chromatography on silica eluting with 5% methanol in dichloromethane containing 0-2% 7N methanol ammonia to give N-(2,3-difluorophenyl)-2-[4-({7-ethoxy-5-[(2R)-piperidin-2-ylmethoxy]quinazolin-4-yl}amino)-1H-pyrazol-1-yl]acetamide (288 mg, 91% yield):

¹H NMR (DMSO d₆): 10.2 (br s, 1H), 9.85 (br s, 1H), 8.43 (s, 1H), 8.28 (s, 1H), 7.72 (s, 1H), 7.71-7.66 (m, 1H), 7.19-7.11 (m, 2H), 6.74 (d, 1H), 6.64 (d, 1H), 5.09 (s, 2H), 4.29-4.11 (m, 4H), 3.15-3.09 (m, 1H), 3.08-3.02 (m, 1H), 2.72-2.66 (m, 1H), 1.86-1.80 (m, 1H), 1.71-1.65 (m, 1H), 1.60-1.54 (m, 1H), 1.52-1.29 (m, 6H) MS (+ve ESI): 538 (M+H)⁺

Tert-butyl (2R)-2[({4-[(1-{2-[(2,3-difluorophenyl)amino]-2-oxoethyl}-1H-pyrazol-4-yl)amino]-7-ethoxyquinazolin-5-yl}oxy)methyl]piperidine-1-carboxylate, used as starting material, was prepared as follows:

a) To a solution of 3,5-difluorophenol (13.0 g, 0.10 mol) and potassium carbonate (20.9 g, 0.15 mol) in dimethylformamide (200 ml) cooled in an ice/water bath was added diethylsulphate (13.1 ml, 0.10 mol). The mixture was heated to 80° C. for 1.5 hours. Further portions of diethylsulphate (3.3 ml, 25 mmol) and potassium carbonate (5.2 g, 37.5 mmol) were added and the mixture was heated for a further 2 hours. The resulting solution was allowed to cool to room temperature and then poured into water and extracted twice with diethyl ether. The combined diethyl ether extracts were washed with water, dried over magnesium sulphate and then evaporated to leave 1-ethoxy-3,5-difluorobenzene as a yellow oil (13.59 g, 86% yield):

¹H-NMR (DMSO d₆): 6.77-6.65 (m, 3H), 4.06 (q, 2H), 1.32 (t, 3H).

MS (+ve EI): 158 (M^{+})

b) n-Butyl lithium (13.5 ml of a 1.6 M solution in hexanes, 21.6 mmol) was added dropwise to a stirred solution of 1-ethoxy-3,5-difluorobenzene (3.42 g, 21.6 mmol) in tetrahydrofuran (30 ml) at −78° C. under an atmosphere of nitrogen. The mixture was stirred at −78° C. for 2 hours and then excess solid carbon dioxide pellets were added. The reaction mixture was allowed to warm to room temperature and the resulting solution poured into water. The mixture was made basic by the addition of an aqueous solution of sodium hydroxide and the mixture was then extracted with diethyl ether. The mixture was separated and the aqueous layer made acidic by the addition of dilute hydrochloric acid. The mixture was extracted twice with diethyl ether. The combined diethyl ether extracts were washed with water, dried over magnesium sulphate and then evaporated to leave 4-ethoxy-2,6-difluorobenzoic acid as a colourless solid (3.87 g, 89% yield):

¹H-NMR (DMSO d₆): 13.36 (br. s, 1H), 6.82-6.76 (m, 2H), 4.11 (q, 2H), 1.33 (t, 3H).

MS (+ve CI): 203 (M+H)⁺

c) Oxalyl chloride (3.17 ml, 36.4 mmol) was added dropwise to a stirred suspension of 4-ethoxy-2,6-difluorobenzoic acid (3.5 g, 17.3 mmol) and dimethylformamide (5 drops) in dichloromethane (50 ml). The resulting solution was stirred at room temperature for 4 hours and was then evaporated. The residue was dissolved in tetrahydrofuran and then added dropwise to a vigorously stirring 35% aqueous ammonia solution (60 ml). The mixture was filtered and the residue was washed with ice-cold water and then dried under vacuum to leave 4-ethoxy-2,6-difluorobenzamide as a colourless solid (3.23 g, 93% yield):

¹H-NMR (DMSO d₆): 7.91 (br. s, 1H), 7.65 (br. s, 1H), 6.78-6.71 (m, 2H), 4.08 (q, 2H), 1.32 (t, 3H).

MS (+ve EI): 201 (M^{+})

d) To a stirred suspension of 4-ethoxy-2,6-difluorobenzamide (3.18 g, 15.8 mmol) and triethylamine (4.44 ml, 31.6 mmol) in dichloromethane (25 ml) was added, at 0-5° C., trichloroacetyl chloride (1.94 ml, 17.4 mmol). The mixture was stirred at 0-5° C. for 5 minutes. The resulting solution was washed successively with water, dilute hydrochloric acid, dilute to sodium hydroxide solution, dilute hydrochloric acid and finally with water. The organic solution was dried over magnesium sulphate and then evaporated to leave 4-ethoxy-2,6-difluorobenzonitrile as a pale yellow solid (2.56 g 89% yield):

¹H-NMR (DMSO d₆): 7.06-7.03 (m, 2H), 4.17 (q, 2H), 1.34 (t, 3H).

MS (+ve CI): 184 (M+H)⁺

e) A mixture of 4-ethoxy-2,6-difluorobenzonitrile (8.0 g, 44 mmol) in a saturated solution of ammonia in ethanol (270 ml) was heated to 150° C. in an autoclave for 16 hours. The resulting solution was evaporated and the residue dissolved in dichloromethane and then washed with water. The organic solution was dried over magnesium sulphate, concentrated in vacuo and then purified by chromatography on silica eluting with dichloromethane to give 2-amino-4-ethoxy-6-fluorobenzonitrile as a colourless solid (6.07 g, 77% yield):

¹H-NMR (DMSO d₆): 6.32 (s, 2H), 6.14-6.10 (m, 2H), 3.99 (q, 2H), 1.30 (t, 3H).

MS (+ve EI): 180 (M^{+})

f) 2-Amino-4-ethoxy-6-fluorobenzonitrile (2.5 g, 13.9 mmol) was added portionwise, over 20 minutes, to a mixture of formic acid (20 ml) and concentrated sulphuric acid (5 drops) heated at 100° C. The mixture was heated for 5 hours at 100° C. and then allowed to cool to room temperature. The mixture was poured into ice/water (80 ml) and the resulting precipitate was collected by filtration and washed with water followed by diethyl ether and then dried under vacuum to give 7-ethoxy-5-fluoroquinazolin-4(3H)-one as a colourless solid (2.02 g, 70% yield):

¹H-NMR (DMSO d₆): 12.06 (br s. 1H), 8.01 (s, 1H), 6.91-6.85 (m, 2H), 4.17 (q, 2H), 1.36 (t, 3H).

MS (+ve ESI): 209 (M+H)⁺

g) [(2R)-1-benzylpiperidin-2-yl]methanol (678 mg, 3.30 mmol) in dimethylformamide (5 ml) was added dropwise to a suspension of sodium hydride (264 mg, 6.60 mmol of a 60% weight dispersion in mineral oil) in dimethylformamide (5 ml) and the reaction mixture stirred at room temperature for 30 minutes. 7-ethoxy-5-fluoroquinazolin-4(3H)-one (600 mg, 2.9 mmol) was added and the reaction mixture heated to 60° C. After 1 hour a further portion of sodium hydride (264 mg, 6.60 mmol) was added and heating continued for a further 2.5 hours. The resulting solution was poured into 20% aqueous ammonium chloride solution (40 ml) and the resulting precipitate collected by filtration, washed with water and then dried under vacuum to give 5-{[(2R)-1-benzylpiperidin-2-yl]methoxy}-7-ethoxyquinazolin-4(3H)-one (1.05 g, 97% yield):

¹H NMR (DMSO d₆): 7.83 (s, 1H), 7.34-7.16 (m, 5H), 6.63 (d, 1H), 6.50 (d, 1H), 4.32-4.29 (m, 1H), 4.17-4.02 (m, 4H), 3.55 (d, 1H), 2.81-2.75 (m, 1H), 2.27-2.21 (m, 1.95-1.89 (m, 8H), 1.72-1.59 (m, 2H), 1.56-1.34 (m, 7H).

MS (+ve ESI): 394 (M+H)⁺

[(2R)-1-benzylpiperidin-2-yl]methanol, used as starting material, was prepared as follows: Borane-methylsulphide complex (7.31 ml, 76.0 mmol) was added slowly to a solution of (2R)-1-[(benzyloxy)carbonyl]piperidine-2-carboxylic acid (10.0 g, 38.0 mmol) in tetrahydrofuran (100 ml) at room temperature. The resulting solution was heated to reflux for 4 hours then cooled to room temperature and quenched with methanol (8 ml). 2N aqueous HCl (80 ml) was added and the mixture extracted with ethyl acetate. The combined organic extracts were washed with brine, dried over magnesium sulphate, filtered, and then concentrated to leave a colourless oil. The crude product was purified by chromatography on silica eluting with 5% methanol in dichloromethane. The fractions containing product were evaporated and the residue dried at 50° C. under vacuum to give benzyl (2R)-2-(hydroxymethyl)piperidine-1-carboxylate (8.87 g, 94% yield):

¹H NMR (DMSO d₆): 7.40-7.29 (m, 5H), 5.08 (s, 2H), 4.72-4.67 (m, 1H), 4.18-4.11 (m, 1H), 3.93-3.87 (m, 1H), 3.56-3.43 (m, 2H), 2.87-2.81 (m, 1H), 1.79-1.76 (m, 1H), 1.61-1.24 (m, 5H).

MS (+ve ESI): 250 (M+H)⁺

Palladium on carbon (500 mg of 10%) was added to a solution of benzyl (2R)-2-(hydroxymethyl)piperidine-1-carboxylate (5.00 g, 20.06 mmol) in ethyl acetate (70 ml) and the reaction mixture stirred at room temperature under an atmosphere of hydrogen for 4 hours. The mixture was filtered and a new portion of catalyst (300 mg) was added to the filtrate. The mixture was then stirred under an atmosphere of hydrogen for a further 3 hours. The mixture was filtered and the filtrate evaporated to leave a brown oil. The crude product was purified by kugelrohr distillation to give (2R)-piperidin-2-ylmethanol (1.51 g, 65% yield):

¹H NMR (CDCl₃): 3.53-3.48 (m, 1H), 3.36-3.31 (m, 1H), 3.02 (d, 1H), 2.61-2.53 (m, 2H), 2.35 (s, 2H), 1.75-1.73 (m, 1H), 1.56-1.47 (m, 2H), 1.40-1.25 (m, 2H), 1.14-1.03 (m, 1H).

Benzyl bromide (2.34 ml, 19.53 mmol) and potassium carbonate (8.1 g, 58.6 mmol) were added to a solution of (2R)-piperidin-2-ylmethanol (2.25 g, 19.53 mmol) in ethanol (40 ml) and water (6 ml). The resulting solution was stirred and heated to 80° C. for 6.5 hours and then allowed to cool to room temperature. The reaction mixture was concentrated and the residue suspended in water (50 ml) and then extracted with diethyl ether (2×50 ml). The combined organic extracts were dried over magnesium sulphate and then concentrated. The residue was purified by chromatography on silica eluting with 5% methanol in dichloromethane containing 0-2% methanolic ammonia to give [(2R)-1-benzylpiperidin-2-yl]methanol (2.86 g, 71% yield):

¹H NMR (DMSO d₆): 7.32-7.27 (m, 4H), 7.24-7.18 (m, 1H), 4.41 (t, 1H), 4.08 (d, 1H), 3.67-3.62 (m, 1H), 3.47-3.42 (m, 1H), 3.24 (d, 1H), 2.66-2.62 (m, 1H), 2.32-2.26 (m, 1H), 2.01-1.95 (m, 1H), 1.71-1.60 (m, 2H), 1.48-1.41 (m, 1H), 1.39-1.21 (m, 3H).

MS (+ve ESI): 207 (M+H)⁺

h) Pd/C (100 mg) was added to a solution of 5-{[(2R)-1-benzylpiperidin-2-yl]methoxy}-7-ethoxyquinazolin-4(3H)-one (1.00 g, 2.54 mmol) and di-tent-butyl dicarbonate (610 mg, 2.80 mmol) in dimethylformamide (15 ml) and the reaction mixture stirred at room temperature under an atmosphere of hydrogen for 4 hours. A further portion of di-tert-butyl dicarbonate (305 mg, 1.4 mmol) was added and stirring was continued for a further 1 hour. The mixture was filtered through celite and the filtrate evaporated. The residue was purified by chromatography on silica eluting with dichloromethane containing 0-5% methanol. The appropriate fractions were combined and evaporated to give tent-butyl (2R)-2-{[(7-ethoxy-4-oxo-3,4-dihydroquinazolin-5-yl)oxy]methyl}piperidine-1-carboxylate (515 mg, 50% yield):

¹H NMR (DMSO d₆): 7.83 (s, 1H), 6.65 (d, 1H), 6.59 (d, 1H), 4.46-4.41 (m, 1H), 4.24-4.06 (m, 4H), 3.90-3.84 (m, 1H), 3.07-2.99 (m, 1H), 2.08-2.02 (m, 1H), 1.73-1.49 (m, 5H), 1.40-1.35 (m, 12H).

MS (+ve ESI): 404 (M+H)⁺

i) Phosphorous oxychloride (0.37 ml, 3.97 mmol) was added to a solution of tert-butyl (2R)-2-{[(7-ethoxy-4-oxo-3,4-dihydroquinazolin-5-yl)oxy]methyl}piperidine-1-carboxy late (500 mg, 1.24 mmol) and N,N-diisopropylethylamine (0.73 ml, 4.21 mmol) in 1,2-dichloroethane (15 ml). The reaction mixture was heated to 85° C. for 1.5 hours and then allowed to cool to room temperature. The resulting solution was concentrated and the residue dissolved in dichloromethane (50 ml) and then washed with saturated aqueous sodium bicarbonate solution (50 ml). The organic phase was separated and dried over magnesium sulphate, filtered, and then evaporated. The residue was purified by chromatography on silica eluting with ethyl acetate to give tert-butyl (2R)-2-{[(4-chloro-7-ethoxyquinazolin-5-yl)oxy]methyl}piperidine-1-carboxylate (375 mg, 72% yield):

MS (+ve ESI): 422 (M+H)⁺

j) 2-(4-amino-1H-pyrazol-1-yl)-N-(2,3-difluorophenyl)acetamide (222 mg, 0.88 mmol) was added to a solution of tert-butyl (2R)-2-{[(4-chloro-7-ethoxyquinazolin-5-yl)oxy]methyl}piperidine-1-carboxylate (370 mg, 0.88 mmol) in propan-2-ol and the mixture heated to 70° C. for 15 minutes and then was allowed to cool to room temperature to afford a thick precipitate. The mixture was diluted with diethyl ether and the precipitate collected by filtration, washed with diethyl ether and then dried under vacuum to give tert-butyl (2R)-2-[({4-[(1-{2-[(2,3-difluorophenyl)amino]-2-oxoethyl}-1H-pyrazol-4-yl)amino]-7-ethoxyquinazolin-5-yl}oxy)methyl]piperidine-1-carboxylate hydrochloride (423 mg, 71% yield):

¹H NMR (DMSO d₆): 8.67 (s, 1H), 8.33 (s, 1H), 7.96 (s, 1H), 7.69-7.64 (m, 1H), 7.15-7.05 (m, 2H), 7.01 (d, 1H), 6.85 (d, 1H), 5.11 (s, 2H), 4.81-4.79 (m, 2H), 4.35 (q, 1H), 4.25 (q, 2H), 3.92-3.86 (m, 1H), 3.10-3.02 (m, 1H), 1.82-1.75 (m, 1H), 1.72-1.57 (m, 4H), 1.42-1.36 (m, 4H), 1.21 (s, 91-1).

MS (+ve ESI): 638

2-(4-amino-1H-pyrazol-1-yl)-N-(2,3-difluorophenyl)acetamide, used as a starting material, was prepared as follows:

a) A solution of 2,3-difluoroaniline (12.9 g, 100 mmol) in diethyl ether (100 ml) was treated with 1M aqueous sodium hydroxide (98 ml, 98 mmol) and stirred vigorously while a solution of chloroacetyl chloride (13.3 g, 117 mmol) in diethyl ether (100 ml) was added dropwise over 20 minutes at 5° C. The mixture was allowed to warm to 20° C. over 1 hour and then ethyl acetate (100 ml) was added. The organic phase was separated and washed with 20% aqueous potassium hydrogen carbonate, dried and then evaporated to leave a white solid. The solid was dissolved in boiling tetrahydrofuran (20 ml) and then diluted with cyclohexane (300 ml) and isohexane (100 ml). The mixture was concentrated to approximately 250 ml, cooled and filtered to give 2-chloro-N-(2,3-difluorophenyl)acetamide as white crystals (18.48 g, 90% yield):

¹H-NMR (DMSO d₆): 10.26 (br s, 1H), 7.67 (m, 1H), 7.19 (m, 2H), 4.36 (s,

MS (+ve ESI): 206, 204 (M+H)⁺

b) A solution of 2-chloro-N-(2,3-difluorophenyl)acetamide (10.28 g, 50 mmol) and 4-bromopyrazole (7.35 g, 50 mmol) in dimethylacetamide (20 ml) was treated with potassium carbonate (8.29 g, 60 mmol) and stirred under nitrogen at 20° C. for 18 hours. The mixture was poured into water (300 ml), filtered and the solid washed with water (500 ml) and air-dried. The solid was dissolved in boiling tetrahydrofuran (80 ml), filtered, diluted with cyclohexane (100 ml) and then evaporated to approximately 100 ml. The resultant slurry was diluted with isohexane (100 ml), cooled and filtered to give 2-(4-bromo-1H-pyrazol-1-yl)-N-(2,3-difluorophenyl)acetamide as a white solid (13.09 g, 83% yield):

¹H-NMR (DMSO d₆): 10.32 (br s, 1H), 8.00 (s, 1H), 7.70 (m, 1H), 7.59 (s, 1H), 7.19 (m, 2H), 5.14 (s, 2H).

MS (+ve ESI): 316, 318 (M+H)⁺

c) A solution of 2-(4-bromo-1H-pyrazol-1-yl)-N-(2,3-difluorophenyl)acetamide (9.48 g, 30 mmol) and (9,9-dimethyl-9H-xanthene-4,5-diyl)bis(diphenylphosphine) (1.74 g, 3 mmol) in anhydrous 1,4-dioxane (50 ml) was treated with tris(dibenzylideneacetone)dipalladium(0) (1.37 g, 1.5 mmol) and the mixture stirred for 5 minutes under nitrogen. Benzophenone imine (5.7 g, 31.5 mmol) was added in one portion, followed by sodium tert-butoxide (8.64 g, 90 mmol). The mixture was degassed with nitrogen and then heated under nitrogen to 90° C. for 4 hours. The mixture was cooled, diluted with diethyl ether (100 ml) and then poured into saturated aqueous ammonium chloride (100 ml). The mixture was filtered through celite and then the layers were separated. The organic phase was dried over magnesium sulphate and concentrated to an oil which was extracted twice with boiling cyclohexane (200 ml, 100 ml). The cyclohexane solution was evaporated to a gum which was crystallized from isohexane:diethyl ether 1:1 to give N-(2,3-difluorophenyl)-2-{4-[(diphenylmethylene)amino]-1H-pyrazol-1-yl}acetamide as a pale yellow solid (5.50 g, 44% yield):

¹H-NMR (DMSO d₆): 10.21 (br s, 1H), 7.66 (m, 3H), 7.56 (m, 3H), 7.44 (m, 3H), 7.35 (s, 1H), 7.24 (m, 2H), 7.18 (m, 2H), 6.48 (s, 1H), 4.98 (s, 2H).

MS (+ve ESI): 417 (M+H)⁺

d) A well stirred solution of N-(2,3-difluorophenyl)-2-{4-[(diphenylmethylene)amino]-1H-pyrazol-1-yl}acetamide (2.08 g, 5 mmol) in ethyl acetate (25 ml) was treated dropwise with 37% aqueous hydrochloric acid (0.496 ml, 6 mmol) over 1 minute at room temperature. The mixture was stirred for 1 hour and then filtered. The residue was washed with ethyl acetate and diethyl ether and then air-dried to leave 2-(4-amino-1H-pyrazol-1-yl)-N-(2,3-difluorophenyl)acetamide hydrochloride as a white powder (1.35 g, 93% yield):

¹H NMR (DMSO d₆): 10.48 (s, 1H); 10.22 (br s, 3H); 8.03 (s, 1H); 7.68 (m, 1H); 7.60 (s, 1H); 7.19 (m, 2H); 5.20 (s, 2H).

MS (+ve ESI): 253 (M+H)⁺

e) 2-(4-amino-1H-pyrazol-1-yl)-N-(2,3-difluorophenyl)acetamide hydrochloride (1.685 g, 5.84 mmol) was suspended in a mixture of ethyl acetate (70 ml) and saturated aqueous sodium bicarbonate (35 ml) and then stirred for 1 hour. The clear layers were separated and the aqueous phase washed with ethyl acetate (4×30 ml). The combined organic solutions were dried over magnesium sulphate and then evaporated to a solid which was washed with diethyl ether giving 2-(4-amino-1H-pyrazol-1-yl)-N-(2,3-difluorophenyl)acetamide as a pink solid (1.377 g, 94%).

¹H NMR (DMSO d₆): 10.06 (br s, 1H); 7.70 (m, 1H); 7.17 (m, 2H); 7.08 (s, 1H); 6.98 (s, 1H): 4.90 (s, 2H); 3.84 (br s, 2H).

MS (+ve ESI): 253 (M+H)⁺

### EXAMPLE 2

### Preparation of Compound 2 in Table 1—N-(2,3-difluorophenyl)-2-[4-({7-methoxy-5-[(2R)-piperidin-2-ylmethoxy]quinazolin-4-yl}amino)-1H-pyrazol-1-yl]acetamide

An analogous reaction to that described for example 1 but starting with tert-butyl (2R)-2-[({4-[(1-{2-[(2,3-difluorophenyl)amino]-2-oxoethyl}-1H-pyrazol-4-yl)amino]-7-methoxyquinazolin-5-yl}oxy)methyl]piperidine-1-carboxylate hydrochloride (325 mg, 0.49 mmol) yielded compound 2 in Table 1 (210 mg, 81% yield):

¹H NMR (DMSO d₆): 10.40 (s, 1H), 10.28 (s, 1H), 8.47 (s, 1H), 8.36 (s, 1H), 7.72 (m, 1H), 7.67 (s, 1H), 7.24-7.16 (m, 2H), 6.76 (d, 1H), 6.68 (d, 1H), 5.15 (s, 2H), 4.27 (m, 1H), 4.10 (m, 1H), 3.89 (s, 3H), 3.13 (m, 1H), 2.99 (m, 1H), 2.67 (t, 1H), 1.86-1.77 (m, 1H), 1.68-1.52 (m, 2H), 1.51-1.24 (m, 3H).

MS (+ve ESI): 524 (M+H)⁺.

tent-Butyl (2R)-2-[({4-[(1-{2-[2,3-difluorophenyl)amino]-2-oxoethyl}-1H-pyrazol-4-yl)amino]-7-methoxyquinazolin-5-yl}oxy)methyl]piperidine-1-carboxylate, used as starting material, was prepared as follows:

a) An analogous reaction to that described in example 1f but starting with 2-amino-6-fluoro-4-methoxybenzonitrile (1.3 g, 7.83 mmol) gave 5-fluoro-7-methoxyquinazolin-4(3H)-one (1.15 g, 76% yield):

¹H NMR (DMSO d₆): 12.08 (s, 1H), 8.02 (s, 1H), 6.94 (m, 1H), 6.90 (d, 1H), 3.89 (s, 3H).

MS (+ve ESI): 195 (M+H)⁺.

2-Amino-6-fluoro-4-methoxybenzonitrile is described in WO 03/076427 and the process for its preparation described therein is incorporated herein by reference.
b) An analogous reaction to that described in example 1 g but starting with 5-fluoro-7-methoxyquinazolin-4(3H)-one (500 mg, 2.58 mmol) gave 5-{[(2R)-1-benzylpiperidin-2-yl]methoxy}-7-methoxyquinazolin-4(3H)-one (828 mg, 85% yield):

MS (+ve ESI): 380 (M+H)⁺.

c) An analogous reaction to that described in example 1 h but starting with 5-{[(2R)-1-benzylpiperidin-2-yl]methoxy}-7-methoxyquinazolin-4(3H)-one (828 mg, 2.18 mmol) gave tert-butyl (2R)-2-{[(7-methoxy-4-oxo-3,4-dihydroquinazolin-5-yl)oxy]methyl}piperidine-1-carboxylate (606 mg, 71% yield):

MS (+ve ESI): 390 (M+H)⁺.

d) An analogous reaction to that described in example 11 but starting with tert-butyl (2R)-2-{[(7-methoxy-4-oxo-3,4-dihydroquinazolin-5-yl)oxy]methyl}piperidine-1-carboxylate (606 mg, 1.56 mmol) gave tert-butyl (2R)-2-{[(4-chloro-7-methoxyquinazolin-5-yl)oxy]methyl}piperidine-1-carboxylate (506 mg, 80% yield).
e) An analogous reaction to that described in example 1j but starting with tert-butyl (2R)-2-{[(4-chloro-7-methoxyquinazolin-5-yl)oxy]methyl}piperidine-1-carboxylate (253 mg, 0.62 mmol) gave tert-butyl (2R)-2-[({4-[(1-{2-[(2,3-difluorophenyl)amino]-2-oxoethyl}-1H-pyrazol-4-yl)amino]-7-methoxyquinazolin-5-yl}oxy)methyl]piperidine-1-carboxylate hydrochloride (325 mg, 79% yield):

MS (+ve ESI): 624 (M+H)⁺.

1. A compound of formula (I) 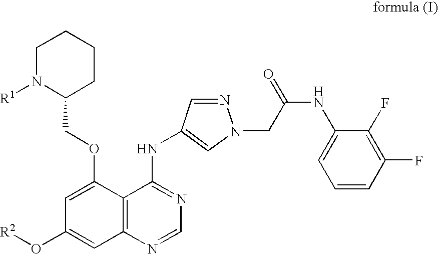
or a pharmaceutically acceptable salt thereof;
wherein R¹ is hydrogen or methyl; and
R² is methyl or ethyl.

2. A compound according to claim 1 wherein R² is methyl.

3. A compound according to claim 1 wherein R² is ethyl.

4. A compound according to claim 1 wherein R¹ is hydrogen.

5. A compound according to claim 1 wherein R¹ is methyl.

6. A compound according to claim 1 selected from N-(2,3-difluorophenyl)-2-[4-({7-methoxy-5-[(2R)-piperidin-2-ylmethoxy]quinazolin-4-yl}amino)-1H-pyrazol-1-yl]acetamide and N-(2,3-difluorophenyl)-2-[4-({7-methoxy-5-[(2R)-1-methylpiperidin-2-ylmethoxy]quinazolin-4-yl}amino)-1H-pyrazol-1-yl]acetamide or a pharmaceutically acceptable salt thereof.

7. A compound according to claim 1 selected from N-(2,3-difluorophenyl)-2-[4-({7-ethoxy-5-[(2R)-piperidin-2-ylmethoxy]quinazolin-4-yl}amino)-1H-pyrazol-1-yl]acetamide and N-(2,3-difluorophenyl)-2-[4-({7-ethoxy-5-[(2R)-1-methylpiperidin-2-ylmethoxy]quinazolin-4-yl}amino)-1H-pyrazol-1-yl]acetamide or a pharmaceutically acceptable salt thereof.

8. A pharmaceutical composition comprising a compound according claim 1, in association with a pharmaceutically acceptable diluent or carrier.

9-11. (canceled)

12. A method of treating a human suffering from a disease in which the inhibition of one or more aurora kinase is beneficial, comprising the steps of administering to a person in need thereof a therapeutically effective amount of a compound according to claim 1.

13. A method of treating a human suffering from a hyperproliferative disease, comprising the steps of administering to a person in need thereof a therapeutically effective amount of a compound according to claim 1.

14. A process for the preparation of a compound according to claim 1 where R¹ is methyl, which process comprising reacting a compound of formula (I) where R¹ is hydrogen with formaldehyde in formic acid at elevated temperatures from 50° C. to 100° C. for 30 minutes to 2 hours, and thereafter if necessary:
i) removing any protecting groups; and/or
ii) forming a pharmaceutically acceptable salt thereof.

15. A process for the preparation of a compound according to claim 1 wherein R¹ is hydrogen comprising reacting a compound of formula (II): 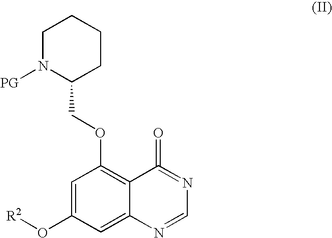
where PG is a suitable protecting group and L is a suitable leaving group, with 2-(4-amino-1H-pyrazol-1-yl)-N-(2,3-difluorophenyl)acetamide and thereafter if necessary:
i) removing any protecting groups; and/or
ii) forming a pharmaceutically acceptable salt thereof.

16. The method of claim 13, wherein the hyperproliferative disease is cancer.

17. The method of claim 16, wherein the cancer is any one of or any combination of, colorectal, breast, lung, prostate, bladder, renal or pancreatic cancer or leukaemia or lymphoma.

18. The process of claim 15, wherein PG is tert-butoxycarbonyl (BOC), benzyloxycarbonyl (Z) or 9-fluorenylmethyloxycarbonyl (Fmoc).

19. The process of claim 15, wherein L is chloro.
